(19) United States
(12) Patent Application Publication
(10) Pub. No.: US 2005/0004465 A1
(43) Pub. Date: Jan. 6, 2005

(54) System, method and medium for generating operator independent ultrasound images of fetal, neonatal and adult organs

(75) Inventor: Alfred Z. Abuhamad, Virginia Beach, VA (US)

Correspondence Address: WILMER CUTLER PICKERING HALE AND DORR LLP, THE WILLARD OFFICE BUILDING, 1455 PENNSYLVANIA AVE, NW, WASHINGTON, DC, 20004, US

(73) Assignee: Eastern Virginia Medical School, Norfolk, VA (US)

(21) Appl. No.: 10/823,793

(22) Filed: Apr. 14, 2004

Related U.S. Application Data
(60) Provisional application No. 60/463,045, filed on Apr. 16, 2003.

(51) Int. Cl. A61B 8/00

(52) U.S. Cl. 600/443

(57) ABSTRACT

A system, method and medium, for use in a medical-imaging environment, that acquires ultrasound image data for at least a portion of a body organ, utilizes data defining a reference plane for the body organ to define at least one other plane with respect to the reference plane, and displays ultrasound images corresponding to at least one of the reference plane and the data defining the at least one other plane.

### CROSS-REFERENCE TO RELATED APPLICATION

This application claims priority to, and incorporates by reference, Provisional Application Ser. No. 60/463,045, filed Apr. 16, 2003.

### DESCRIPTION

### BACKGROUND OF THE INVENTION

1. Field of the Invention

The present invention generally relates to generating ultrasound still and/or real time images and, more particularly, to generating operator-independent displays of ultrasound still and/or real time images of standard anatomic planes of fetal, neonatal and/or adult organs.

2. Background Description

Ultrasonography is an operator dependent imaging modality. That is, unlike other imaging techniques such as computed tomography (CT) and magnetic resonance imaging (MRI), the quality of the image(s) provided by ultrasound technology depends directly on the skills of the sonographer and/or the sonologist obtaining the images. Furthermore, in obstetrical ultrasound imaging, the variable position of the fetus within the uterus is an added factor that raises the level of difficulty.

Several studies have documented that the efficacy of ultrasonography, especially with regard to the detection of fetal abnormalities, is dependent on the expertise of the operator. See, Ewigman B. G., Crane J. P., Frigoletto F. D., Leferve M. L., Bain R. P., McNellis D., Effect of Prenatal Ultrasound Screening on Perinatal Outcome, The RADIUS Study Group, New England Screening on Perinatal Outcome, The RADIUS Study Group, New England Journal of Medicine, 1993; 171:821-827; Chitty L. S., Ultrasound Screening for Fetal Abnormalities, Prenatal Diagnosis, 1995; 15:1241-57; Crane J. P., LeFerve M. L., Winbron R. C., et al., A Randomized Trial of Prenatal Ultrasonographic Screening: Impact on the Detection, Management, and Outcome of Anomalous Fetuses, The RADIUS Study Group, American Journal of Obstetrics and Gynecology, 1994; 171:392-399; Grandjean H., Larroque D., Levi S., and the Eurofetus Study Group, American Journal of Obstetrics and Gynecology, 1999; 181:446-454.

Studies performed in the United States and Europe reported on a significant difference in the detection of fetal abnormalities on obstetrical ultrasonography between tertiary and non-tertiary centers. See, Ewigman et al., and Chitty. It is generally believed that many women in the United States today receive an obstetrical ultrasound examination that is lower in standards than currently recommended by various professional societies. See, Filly R. A. and Crane J. P., Routine Obstetric Sonography, Journal of Ultrasound Medicine 2002; 21:713-718.

In the art of medical imaging, the development of three-dimensional (3-D) and four-dimensional (4-D) ultrasonography provides an advance in imaging technology. With 3-D ultrasonography, an infinite number of two-dimensional (2-D) planes are acquired of (or within) a target volume. The volume acquired by 3-D ultrasonography can be displayed on a display monitor by the three orthogonal planes representing the sagittal (front/back), transverse (left/right) and coronal (top/bottom) planes of a representative 2-D plane within this volume. Such display of an acquired 3-D volume by 3 orthogonal planes is known as multiplanar imaging (or multiplanar display). The multiplanar display of ultrasound volumes enables an operator to manipulate the acquired target volume. In the multiplanar display, the volume can be explored by scrolling through parallel planes in any of the three views, and by rotating the volume to obtain a view of the structures of interest. The operator can thus manipulate the volume data to obtain any desired plane of section after the volume is acquired and the patient is discharged. Thus, one advantage of 3-D ultrasound is the ability to obtain different views from one stored volume. Conventional 3-D technology allows for a display of a cineloop of a full cardiac cycle when imaging the fetal heart in a multiplanar display. As used herein, a cineloop generally acquires images from many cardiac cycles (typically 10-60), and the resulting time series of images is averaged over many cardiac cycles. When played in a loop, the images demonstrate the moving heart in a movie format. Color flow Doppler can be added, thus allowing for a display of blood flow across the heart valves in the fetus. With 4-D ultrasonography, time is added as the fourth dimension to provide real time (or near real time) display of the surface of the 3-D volume under examination. Even for trained personnel, 3-D volume manipulation by the multiplanar display process can be difficult to perform, particularly when the volume involves relatively complex anatomical organs, such as the central nervous system or the heart.

The professional literature to date pertaining to 3-D ultrasonography generally indicates that 3-D ultrasonography provides diagnostic capabilities beyond those of 2-D ultrasonography. The literature also generally indicates that 3-D ultrasonography provides better visualization of anatomical structures than does 2-D ultrasonography. However, some skepticism exists with regard to the real value of 3-D ultrasonography, and whether 3-D ultrasonography improves the diagnostic capabilities and efficacy of current 2-D systems.

There are known ultrasound imaging systems that enable, for example, imaging personnel, such as a sonography technician, to select one or more pre-set anatomical views. For example, U.S. Pat. No. 6,174,285 (the '285 patent) is primarily directed to providing specific planes (views) of the adult heart that cannot be imaged by using conventional 2-D ultrasonography. In 2-D ultrasonography, certain views of the adult heart are unavailable due to it being surrounded by, for example, dense bony structures and air-filled lung tissue.

However, the '285 patent cannot be utilized in connection with obstetrical ultrasound, particularly since the '285 patent indicates that the ultrasound transducer is placed on the patient in standard locations and/or orientations. The '285 patent is thus dependent on and limited by the ultrasound transducer having to be placed in a particular position to make an initial acquisition of a 3-D volume. Moreover, the '285 patent is limited to the user selecting a pre-set anatomical view, and does not contemplate automatically displaying two or more standardized reference planes of interest for a particular body organ. Nor does the '285 patent suggest the desirability of providing a diagnostic capability.

In contrast to ultrasound imaging of the adult heart, standard ultrasound transducer imaging positions on the maternal abdomen are not possible or available in obstetrical ultrasound imaging due, for example, to variable fetal positions within the uterus. Accordingly, personnel acquiring images of the fetal heart cannot rely on standard transducer positions (e.g., a particular position and/or orientation on the maternal abdomen). Instead, imaging personnel are required to dynamically position the transducer in different positions and/or planes until desired images are acquired. It is due at least in part to this difference in scanning techniques between obstetrical ultrasonography and other ultrasound modalities that makes the former difficult to master.

### SUMMARY OF THE INVENTION

It is a feature and advantage of the present invention to provide a system, method and medium of generating operator independent ultrasound display of fetal, neonatal and/or adult organs.

It is still another feature and advantage of the present invention to provide a system, method and medium that utilizes operator independent ultrasound display of standard anatomic planes of fetal, neonatal and/or adult organs to detect normal and/or abnormal imaging relationships within the organ.

It is yet another feature and advantage of the present invention to provide a system, method and medium that improves the efficiency and diagnostic capabilities of current ultrasound examinations of fetal, neonatal and/or adult organs.

It is a further feature of the present invention to facilitate sonography-related teaching and education, and facilitate training of various medical personnel.

At least one embodiment of the present invention can utilize, for example, a computer program in conjunction with, for example, a general purpose computer and/or standard sonography equipment to obtain and optionally display 2-D, 3-D and/or 4-D ultrasound images. In addition, at least one embodiment of the present invention can provide a medical evaluation or diagnosis of aspects of fetal, neonatal and adult organs (e.g., the fetal heart).

In an exemplary method in accordance with the present invention, a reference plane is obtained for a particular body organ, which can be used as a baseline from which to obtain other planes of interest, such as the four-chamber view plane of the fetal heart. The reference plane can optionally be a standard representative plane that is relatively easy to obtain on 2-D ultrasonography, such as the four-chamber view plane of the fetal heart. Exemplary reference planes for the fetal head are the axial biparietal diameter, the axial posterior fossa, the axial lateral ventricles, and the coronal corpus callosum.

A 3-D ultrasound imaging apparatus can then be used to acquire a volume of tissue starting, for example, from the level of (or with respect to) the reference plane. The multiplanar display of this acquired volume shows the reference plane in one of the three displayed orthogonal planes, typically in the A plane (current standard 3-D acquisition). In accordance with at least one embodiment of the present invention, the spatial mathematical relationship of standardized planes in relation to the reference plane are provided for various fetal, neonatal and adult organs. Software and/or hardware utilized by a general purpose computer and/or standard sonography equipment may then utilize one or more of the mathematical relationships, optionally automatically, to display one or more of the standardized planes. In at least one embodiment of the invention, all standardized planes of interest for a particular body organ may be displayed. Further, either a multiplanar display (where one view of the three-plane multiplanar display is a standardized plane), or a display that shows only one or more standardized planes (without any non-standardized planes that may be part of the multiplanar view), may be provided. As transducer and/or processing capability permit, at least one embodiment of the invention can automatically display one or more standardized planes for a body organ in real time (or substantially in real time), thus bypassing the multiplanar display upon obtaining a scanned volume for the body part.

Advantageously, the constant anatomic relationship of these standardized planes to each other will allow the standardized planes to be used on any patient. In the case of fetal organs, slight modification with regard to the gestational age of the fetus may be utilized to facilitate display. The process of displaying all standardized planes of a particular organ is an operator-independent method of evaluating the organ by ultrasound. In at least one embodiment of the invention, the operator also has the option of viewing a real time display of the standardized planes that are automatically generated.

In at least one embodiment of the present invention, computerized diagnostic capabilities can be used to evaluate images associated with one or more of the standardized planes. For example, imaging software can be utilized to recognize a specific structure within an image (representing, e.g., a portion of the fetal heart), compare the image to a reference image, and identify, for example, normal and abnormal anatomical structures and/or portions thereof. Imaging software for the fetal heart can recognize, for example, in one or more planes, the size of the ventricles and/or the outflow tracts, blood flow across various valves within the heart, and generate indicia (e.g., a report) of normal and abnormal relationships. In addition, imaging software can also be used to adjust plane levels to ensure that an optimum or suitable plane is displayed, thus reducing error.

Embodiments of the system, method and medium in accordance with the present invention can provide an image segmentation capability, and orientation tools such as point-to-point references between 2-D and 3-D images that make images easier to interpret and/or enable, for example, diagnostic information to be easily and clearly conveyed to referring physicians and patients. In addition, embodiments of the system, method and medium in accordance with the present invention can provide, for example, volume and weight estimations of the fetus that are based on 3-D volumes (not just 2-D planes).

The present invention thus advantageously and generally improves the diagnostic acumen of ultrasound imaging by both standardizing images and substantially reducing or eliminating the possibility of human error. By substantially reducing or eliminating the impact of the operator, the present invention also improves the efficiency of ultrasound imaging by reducing the time needed to complete an ultrasound examination, thereby resulting in increased throughput and efficiency of ultrasound laboratories.

### BRIEF DESCRIPTION OF THE DRAWINGS

FIG. 1 is a block diagram of an exemplary sonography system that can be used in conjunction with the present invention;

FIG. 2 is a flow diagram of an exemplary method in accordance with the present invention.

FIG. 3 shows a plurality of exemplary standard planes of a fetal heart that can be generated.

FIG. 4 shows an exemplary 3-D multiplanar imaging of a volume of the fetal heart at 20 weeks of gestation, where plane A represents the four-chamber view.

FIG. 5 shows an exemplary 3-D multiplanar imaging of a volume of the fetal heart at 20 weeks of gestation, where plane A represents the right ventricular outflow tract.

FIG. 6 shows an exemplary 3-D multiplanar imaging of a volume of the fetal heart at 20 weeks of gestation, where plane A represents the left ventricular outflow tract.

FIG. 7 shows an exemplary 3-D multiplanar imaging of a volume of the fetal heart at 20 weeks of gestation, where plane A represents the ductal arch.

FIG. 8 shows an exemplary 3-D multiplanar imaging of a volume of the fetal heart at 20 weeks of gestation, where plane A represents the aortic arch.

FIG. 9 shows an exemplary 3-D multiplanar imaging of a volume of the fetal heart at 20 weeks of gestation, where plane A represents the venous connections.

FIG. 10 shows an exemplary 3-D multiplanar imaging of a volume of the fetal heart at 20 weeks of gestation, where plane A represents the three vessel view.

FIG. 11 shows various views that can be generated from a volume of a fetal heart using an alternate scanning technique of standardized transverse views of the fetal abdomen and chest.

### DETAILED DESCRIPTION OF A PREFERRED EMBODIMENT OF THE INVENTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the invention be regarded as including equivalent constructions to those described herein insofar as they do not depart from the spirit and scope of the present invention.

Two concepts of 3-D imaging are pertinent with regard to the present invention. First, the acquired volume of a particular anatomical structure by 3-D ultrasonography, such as a volume of the fetal heart, contains all of the anatomical 2-D planes for a complete evaluation of this structure in normal and abnormal conditions. Second, for every human organ, the anatomical 2-D planes needed to perform a complete anatomical evaluation of a particular organ are organized in a constant anatomic relationship to each other. I have discovered that it is therefore possible to obtain a volume of a specific organ, such as the fetal heart, and utilize an optionally automated software program to display from this volume, one or more 2-D planes that facilitate evaluation of the organ. This aspect of the present invention is referred to as Automated Multiplanar Imaging (AMI). I have further discovered that one or more standardized planes for a particular body organ can be displayed subsequent to acquisition of image data corresponding to the body organ.

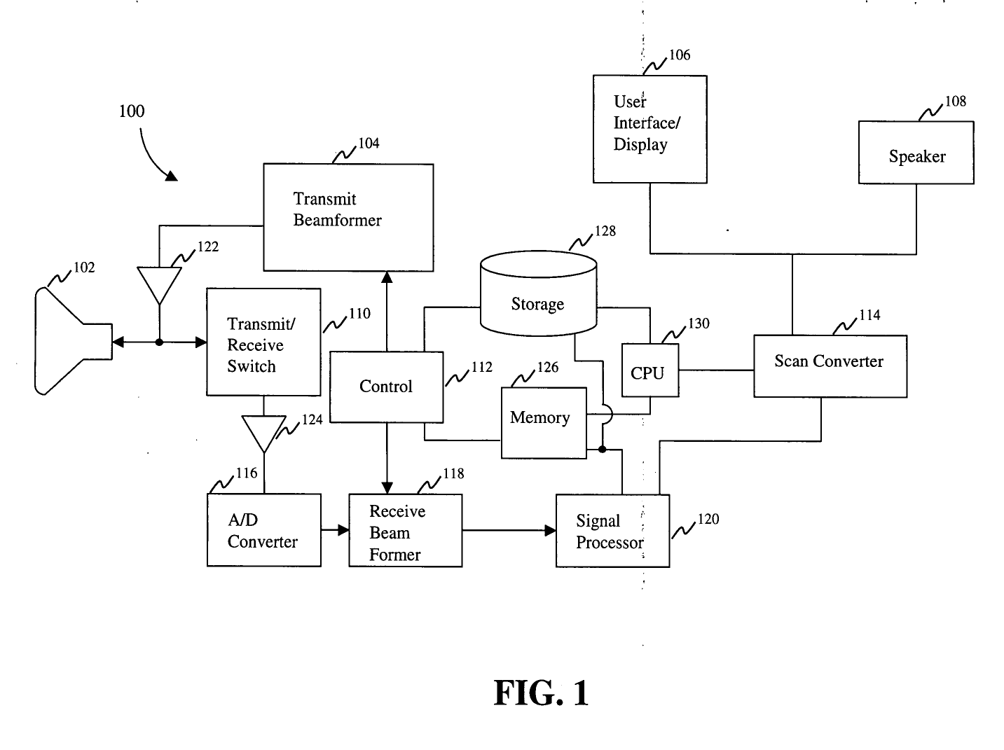

FIG. 1, generally at 100, is a block diagram of an exemplary sonography system that can be used in conjunction with one or more embodiments of the present invention. Transducer 102 is used to scan a volume of a patient's body, to obtain an image of the scanned volume. As known in the art, transducer 102 generally includes a plurality of transducer elements that generate focused acoustic signals responsive to signals generated by transmit beamformer 104. Transducer 102 may include sufficient electronics and/or processing capability to provide or facilitate display of one or more standardized planes subsequent to acquisition (e.g., in a real time or near-real time manner) of image data for a particular body organ. The outputs of transport beamformer 104 can be amplified by amplifier 122 prior to reaching transducer 102.

Transmit/receive switch 110, which can utilize, for example, a plurality of diodes, blocks the transmit beamformer 104 voltage pulses from being received at amplifier 124, A/D converter 116, and receive beamformer 118. Transmit/receive switch 110 thus protects receive beamformer 118 from being damaged by transmit beamformer 104 transmission pulses. In operation, when a transmit pulse from transmit beamformer 104 is present, the diodes of transmit/receive switch 110 switch on, thus short circuiting receive beamformer 118 to ground, while presenting a high impedance path to transmit beamformer 104. In at least one alternate embodiment of the invention, transmit/receive switch 110 does not need to be utilized if separate transmit and receive transducers (not shown) are respectively connected to transmit beamformer 104 and receive beamformer 118.

Transducer 102 receives the ultrasound energy from points within the patient's body, generally at different times, and converts the received ultrasound energy to transducer signals which may be amplified by amplifier 124, converted to digital signals by A/D converter 116, and received by receive beamformer 118. In another embodiment, beamformer 118 can operate on analog signals, if A/D converter 116 is not utilized.

Signal processor 120 may operate to process signals received from receive beamformer 118 in accordance with one or more of at least three primary image acquisition modes. First, 2-D gray-scale imaging, which is referred to as B-mode. Second, Doppler imaging, which is used for blood flow, and is referred to as F-mode. Third, spectral Doppler imaging, can show blood flow velocities and their frequencies, and is referred to as D-mode. Signal processor 120 generally processes signals received from receive beamformer 118 in a manner that substantially optimizes the signals for output in their selected display mode. Signal processor may also optimize signals for audio output using speaker 108, and store the processed signals in memory 126 and/or storage 128. Memory 126 can be, for example, a random access memory, whereas storage 128 may be a medium such as a standard hard drive and/or CD-ROM.

Scan converter 114 is a standard device that, optionally in conjunction with central processing unit (CPU) 130, changes the scan rate of the signals received from signal processor 120 to a scan rate, such as a standard raster scan rate, that is used by user interface/display 106. Display 106 can optionally provide a user-controlled and operated selector, such as a standard mouse, that allows the user to select one or more planes of interest that can be displayed. The user can optionally select any (or all) standardized planes for a particular body organ to be displayed. In at least one embodiment of the invention, the default mode of operation for system 100 can be to display all standardized planes of interest for a particular body organ, once a reference plane is acquired by system 100. Scan converter 114 can also process signals received from signal processor 114 to that they can audibly be output on speaker 108.

Control system 112 coordinates, for example, operation of transmit beamformer 104, receive beamformer 118, signal processor 120, and related elements of system 100. Memory 126 and storage 128 may be used to store, for example, the software that generates standardized planes of interest in accordance with the present invention, as well as control instructions for controller 112.

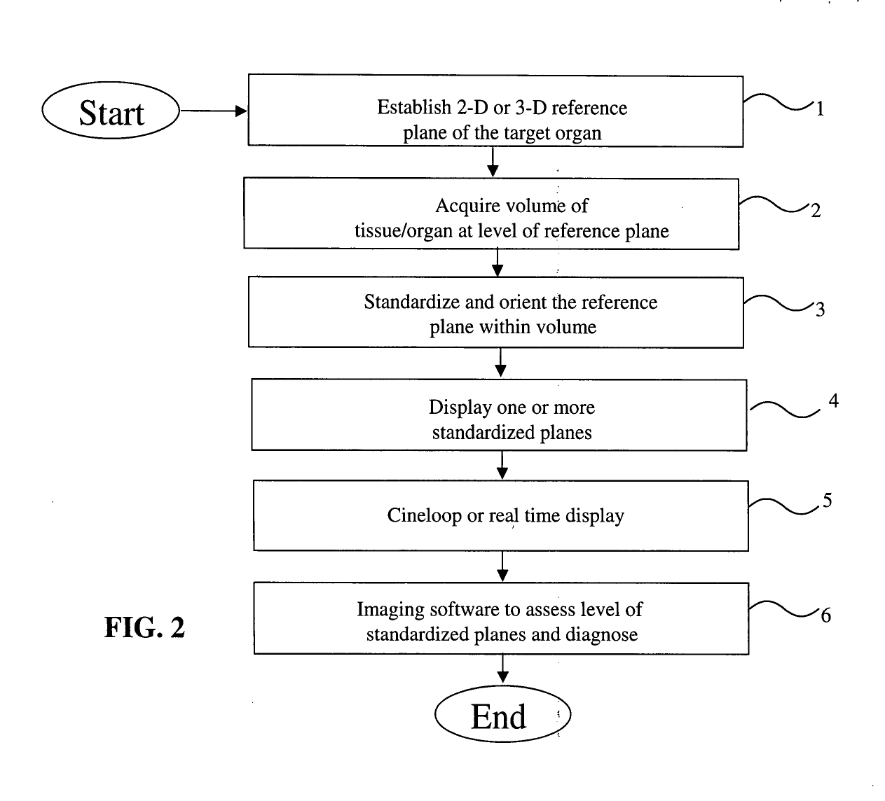

Referring now to FIG. 2, an exemplary method in accordance with the present invention is shown. At step 1, a reference plane is typically obtained in a conventional manner by, for example, a sonographer or a sonologist using conventional 2-D ultrasonography. The reference plane, which is typically a plane that can be readily obtained by 2-D ultrasonography (e.g., four-chamber view of the heart or the biparietal diameter of the head), can be used as a baseline from which to obtain other planes of interest for a particular organ. A sonography system, such as shown in FIG. 1, can be used to obtain the reference plane. The reference plane can also be obtained directly as a volume by 3-D/4-D ultrasonography when transducer technology allows. In general, any plane can be used as a reference plane for a particular organ once the mathematical relationships (e.g., trigonometric relationships) for the standardized planes of interest are defined with respect to a known reference plane. Then, if necessary (or desired), the mathematical relationships for the known reference plane can be adjusted or redefined (e.g., recalculated), using standard mathematical techniques and/or operations for an arbitrary reference plane once the coordinates of the arbitrary reference plane are established. Exemplary planes for the fetal heart that can be utilized are as follows:
a. The four-chamber view
b. The right ventricular outflow
c. The left ventricular outflow
d. The ductal arch
e. The aortic arch
f. The venous connections, and
g. The three vessel view

Planes d, e, and f referred to above are specific fetal cardiac planes that are not ultrasonographically displayed in the adult heart given the presence of air in the adult lungs and the relative large size of the adult heart compared to the fetal heart.

Referring again to FIG. 2, at step 2, a 3-D ultrasound imaging apparatus, such as shown in FIG. 1, can be used to acquire a volume of tissue starting, for example, from the level of the reference plane. The direction of the acquisition is standardized (for example, from abdomen to neck in the case of the fetal heart). In acquiring a volume, position data acquisition may be acquired, for example, by utilizing an integrated positioning system as part of the transducer assembly, or an externally located positioning system.

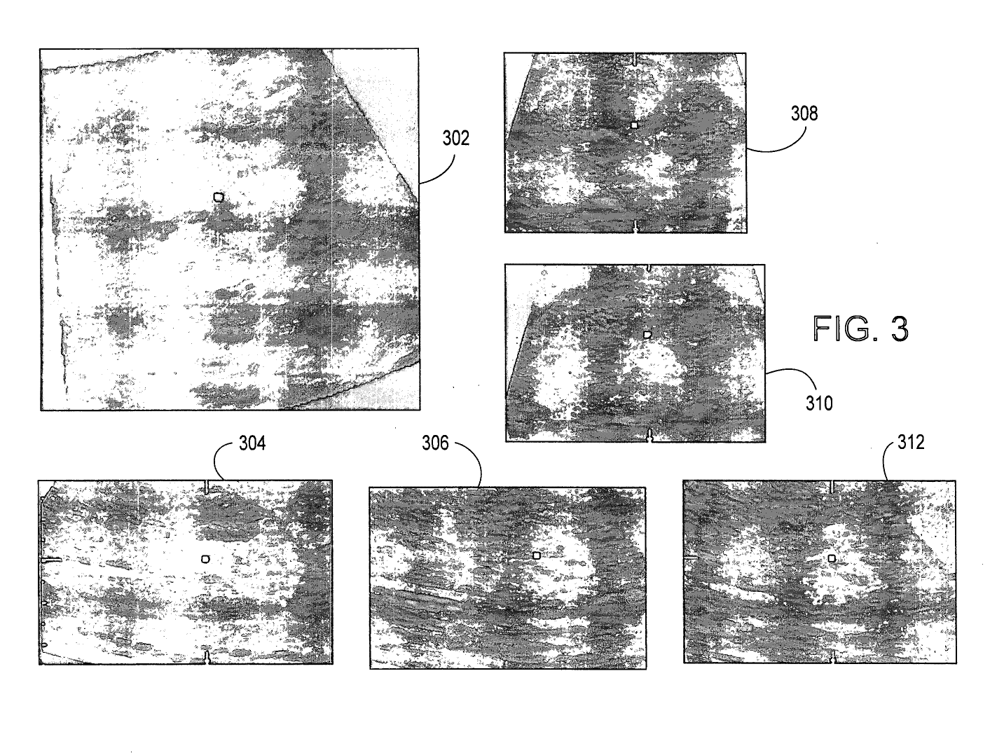

FIG. 3, shows a standard 4-chamber view of a fetal heart 302 that can be used as a reference plane to generate the venous connections view 304, the ductal arch view 306, the left ventricular outflow track 308, the right ventricular outflow track 310, and the aortic arch 312. In another embodiment, one or more of FIGS. 302, 304, 306, 308, 310, 312 can be displayed automatically, subsequent to acquiring the image data. Other standard views for other body organs can also be displayed.

As indicated above, any plane can also be used as a reference plane for the fetal heart, as well as for other organs. For example, a transverse biparietal diameter plane (not shown) can be used as a reference plane of a fetal head.

At step 3 in FIG. 2, the reference plane is fixed and standardized in its orientation within the volume by using standard sonography equipment, such as shown in FIG. 1, to rotate the reference plane into a preset orientation within the volume. For example, the plane of the four-chamber view of the fetal heart 302 can be rotated, using rotation along the Z axis in a standard coordinate system (with the X axis defining the horizontal, and clockwise rotation) to place the spine at approximately the 270° position, and the apex of the heart at approximately the 150° position.

At step 4 in FIG. 2, the computerized program that contains the mathematical formulas that relate the reference plane to all the standardized planes for a particular organ (e.g., the fetal heart) is applied to automatically retrieve one or more of the standardized planes from the acquired 3-D volume. In the case of the fetal heart, once the computerized program is applied to the 3-D volume with the reference plane (e.g., 4 chamber view), any or all planes b-g identified above can be displayed from a single acquisition of the volume as shown in FIG. 3.

Table 1 below describes formulas that can be used to generate standard planes of a fetal heart at approximately 20 weeks of gestation, when the reference plane is the four-chamber view. In a volume, the X, Y, and Z axes represent the three orthogonal axes that are used to define spatial positions within a volume. Any point within a volume can be spatially defined by the X, Y, and Z axes. Furthermore, rotations of planes within a 3-D volume can be performed along the X, Y, and Z axes. The XYZ coordinate system is such that if standard X and Y axes define an XY plane that, for example, divides the front half of the body (or organ) and the back half of the body (or organ), then the Z axis is directed from the front of the body (or organ) to the back of the body (or organ). That is, in this case, a left-handed coordinate system is utilized. Positive rotation is clockwise about an axis.

TABLE 1

| | | Rotation |
|---|---|---|
| Definition | Shift (mm) | (axis, degrees) |
| PA (breech) | 8.2 | 0 |
| PA (cephalic) | −8.2 | 0 |
| Ao (breech) | 3.9 | Y, 27 |
| Ao (cephalic) | −3.9 | Y, 27 |
| 3 vessel (cephalic) | −10.9 | 0 |
| 3 vessel (breech) | 10.9 | 0 |
| Ductal Arch | 0 | Y, 90 |
| (cephalic) | | |
| Aortic Arch | −6.5 | Y, 77 |
| (cephalic) | | |

In the case of Table 1, the reference plane is the four-chamber view. The views determined, and optionally displayed, from the four-chamber view, are shown in the Definition column. The Shift column indicates the shift distance, in millimeters, from (or with respect to) the reference plane. The resulting plane will be parallel to the reference plane, at the specified distance. The Rotation column indicates the number of degrees and the specific axis (X, Y, Z) of rotation. For the standardized plane of the aortic outflow tract for instance, from a four-chamber view reference plane at approximately 20 weeks' gestation, the shift is 3.9 mm in the direction of the fetal head followed by a rotation along the Y axis of 27 degrees, clockwise, when the fetus is in a cephalic presentation and along the Y axis of 27 degrees, counterclockwise, when the fetus is in the breech presentation.

Table 2 below describes additional formulas that can be used to generate standard planes of a fetal heart at approximately 20 weeks of gestation, when the reference plane is the four-chamber view. Transverse views from the fetal abdomen to the neck may be used to allow medical personnel to provide an evaluation of the fetal heart. In this case, the fetal heart can be evaluated when a volume is obtained by sliding transducer 102 transversely (axial plane) from the fetal stomach up to the neck.

TABLE 2

| Definition | Shift (mm) |
|---|---|
| Abdominal | 17.5 |
| circumference | |
| Left ventricular | −3.9 |
| outflow tract (aorta) | |
| Right ventricular | −8.2 |
| outflow tract (PA) | |
| Three vessel view | −10.9 |

Thus, a view of the left ventricular outflow tract can be obtained by shifting a plane—3.9 mm from (e.g., away from the stomach) and parallel with the four-chamber view. In addition, an axial view of the abdomen at the level of the stomach can be obtained by shifting the plane 17.5 mm from the four-chamber view. Note that since the three planes of Table 2 are each transverse planes (i.e., parallel to the four-chamber view), only distance (in mm) is utilized, and rotation about any plane is not required.

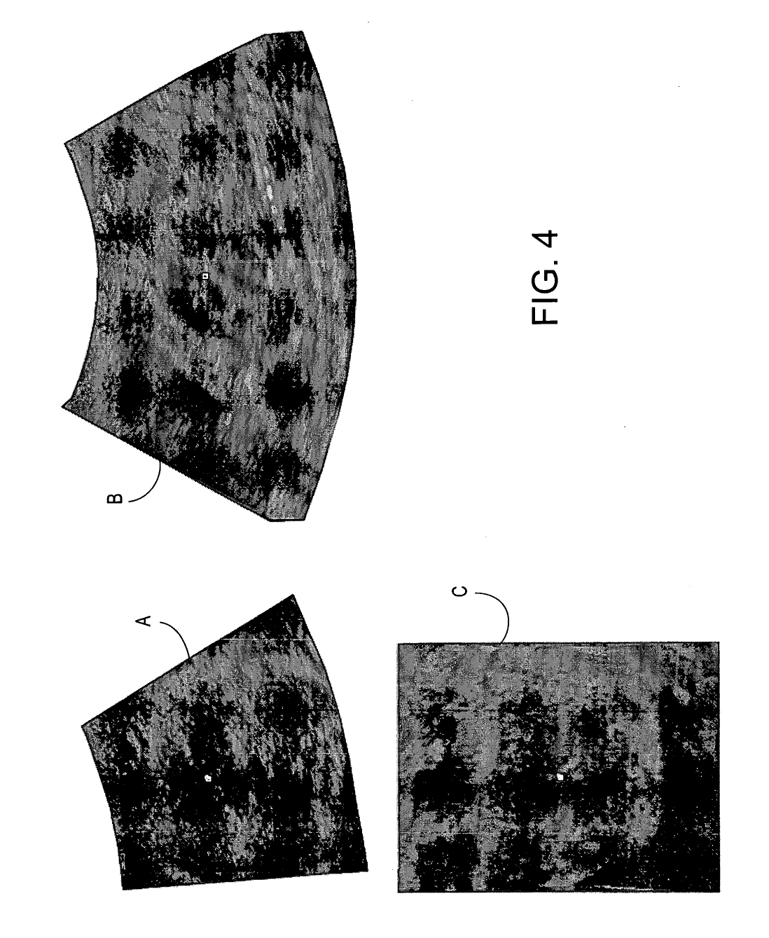

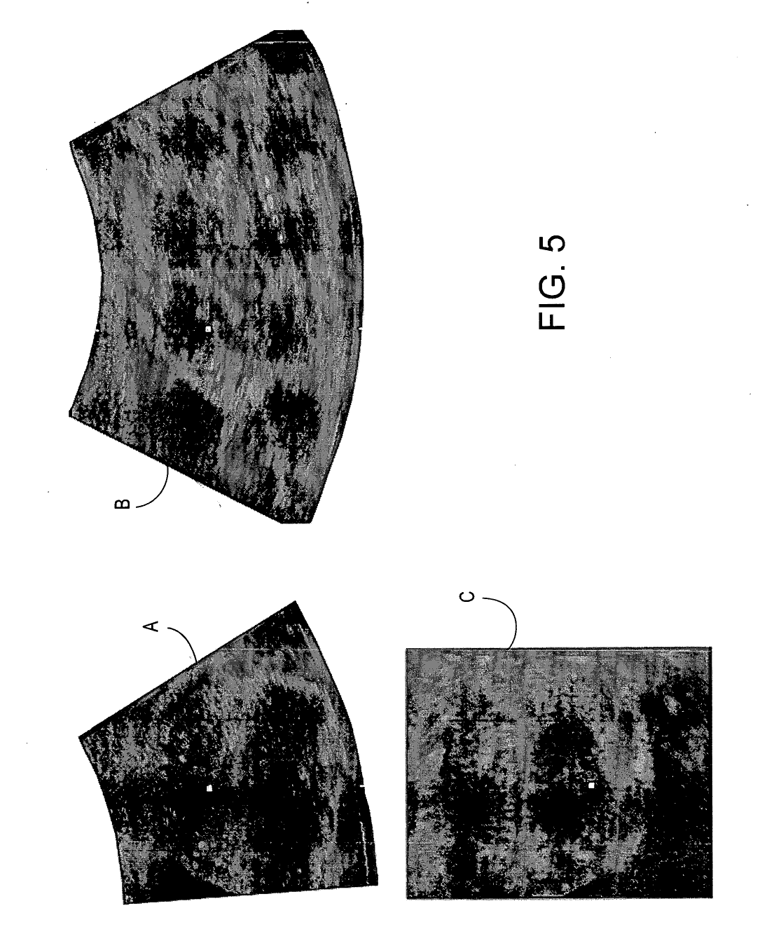

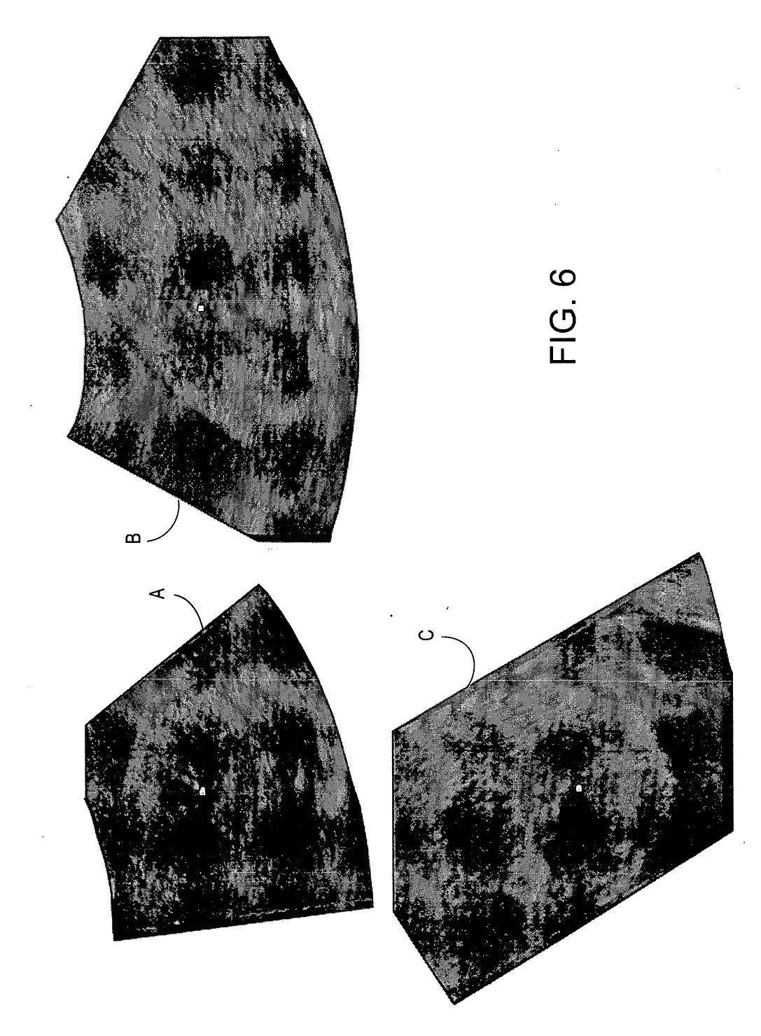

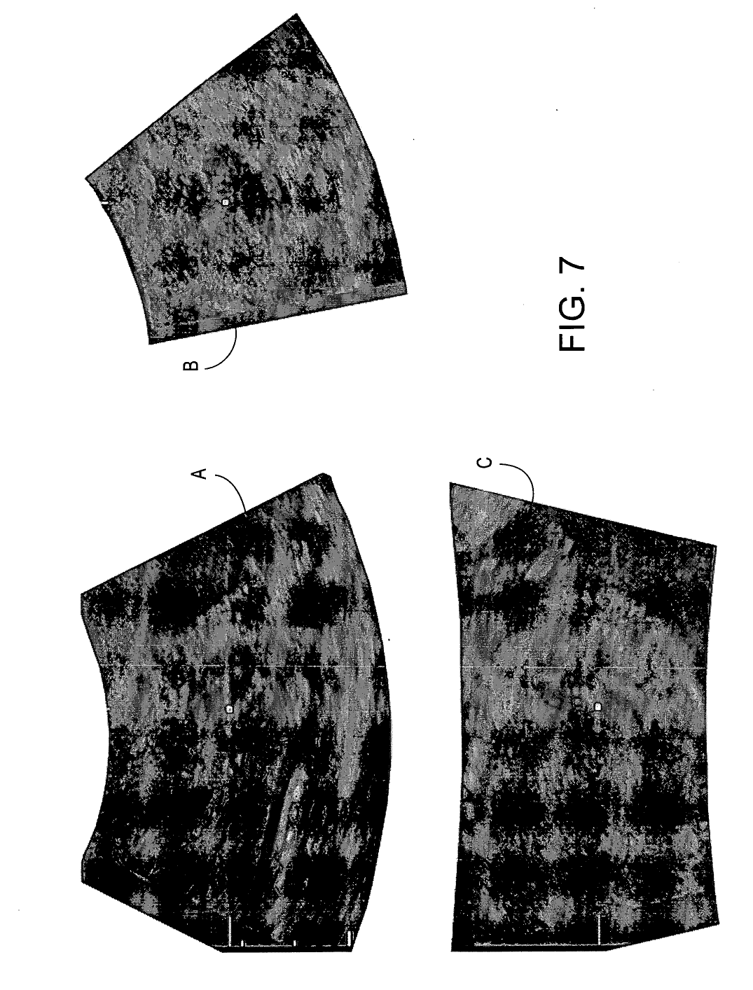

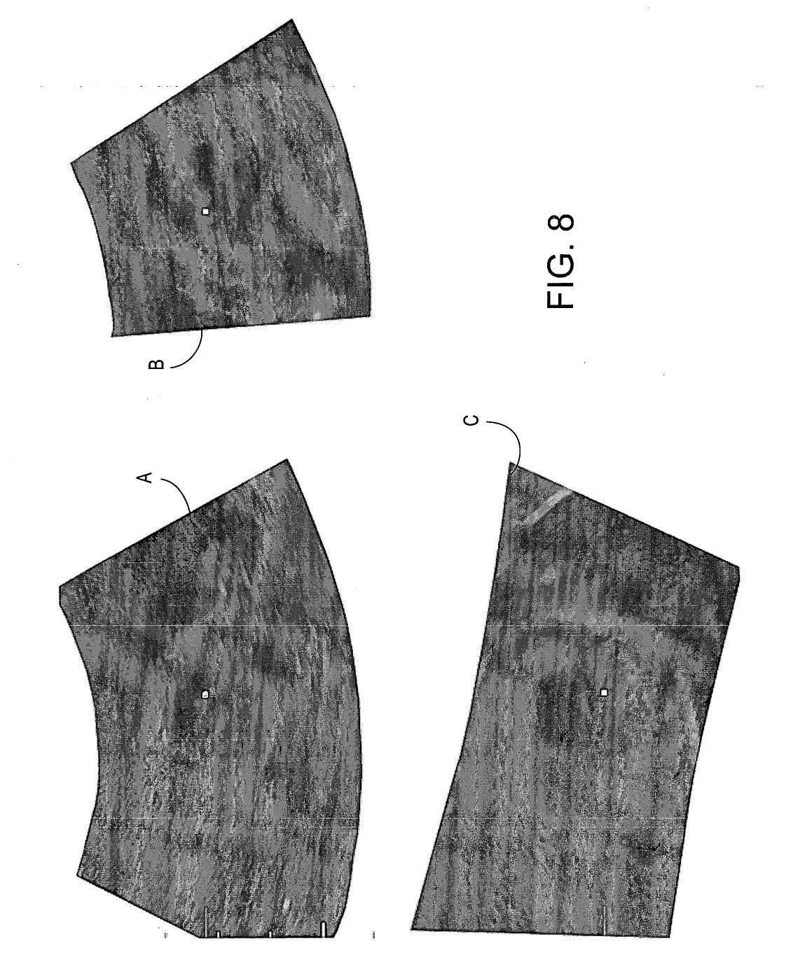

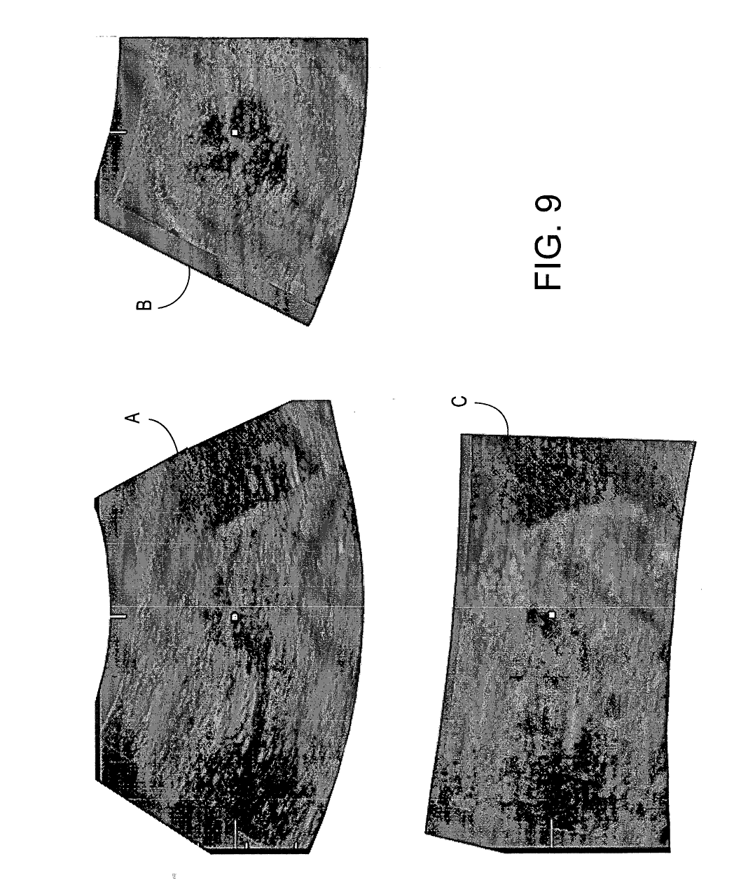

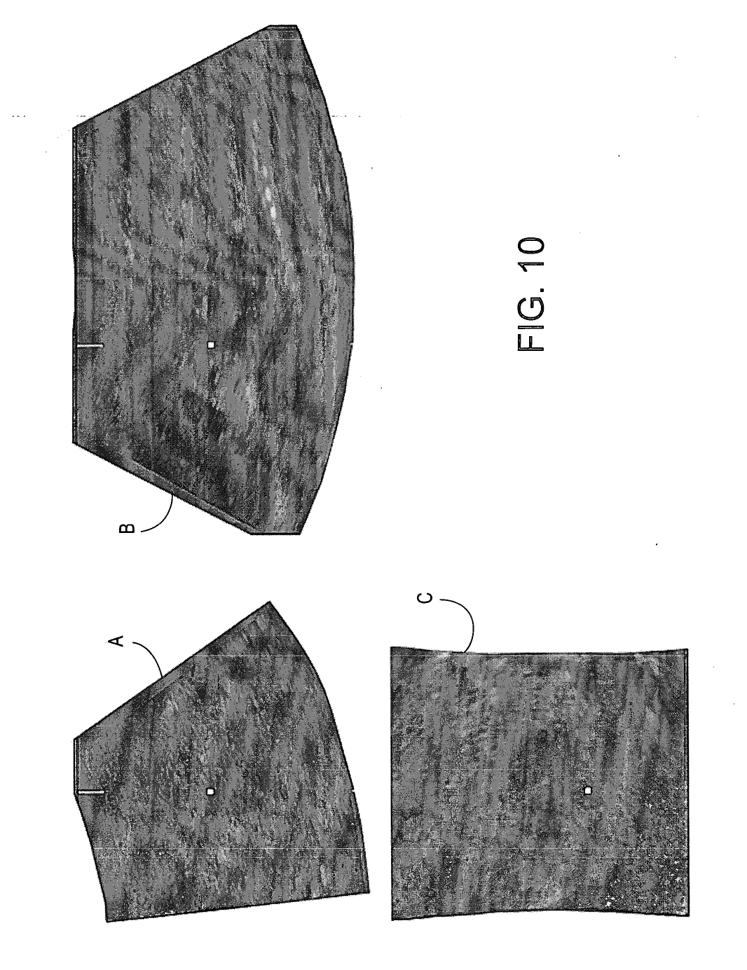

In FIGS. 5-10, 3-D multiplanar displays of the fetal heart are shown, with the A (top left), B (top right) and C (lower left) planes respectively representing the three orthogonal planes for the particular standardized plane (A, top left) at study. In each of FIGS. 5-10, plane A represents a standardized fetal heart plane (b-g listed above), and each standardized plane (A) shown in FIGS. 5-10 is a plane that has been generated, using the mathematical relationships described in Table 1, from the volume displayed in FIG. 4.

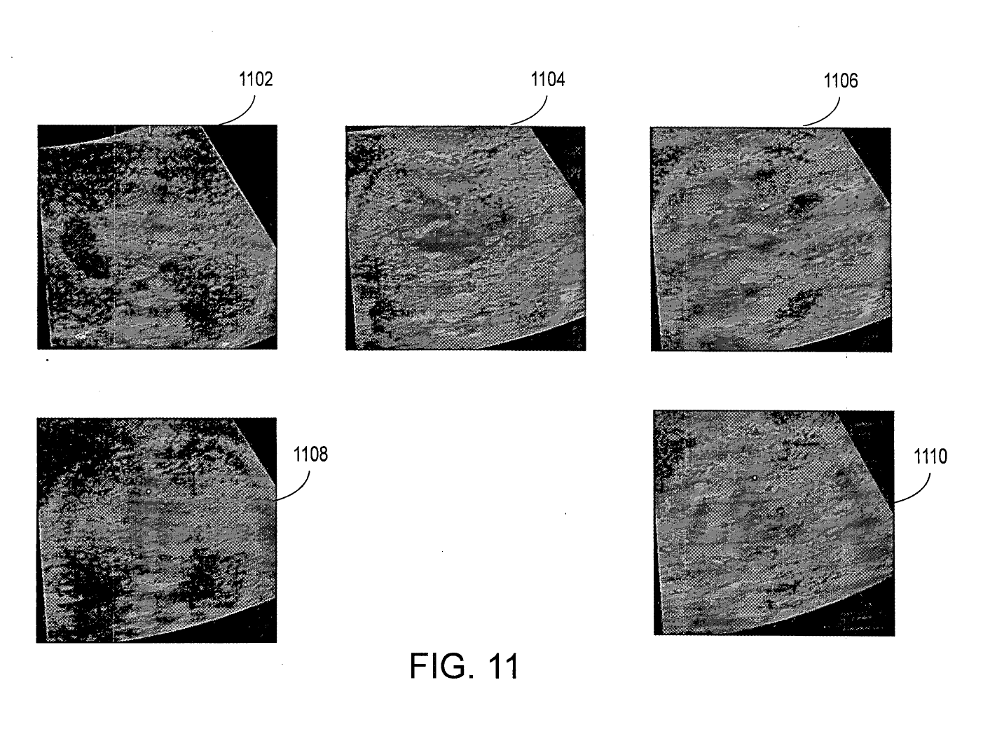

FIG. 11 shows exemplary planes generated in accordance with the techniques described with regard to Table 2. In particular, FIG. 1102 represents an axial plane of the abdomen at level of the fetal stomach (shifted 17.5 mm from the four-chamber view), and FIG. 1104 shows the four-chamber view. FIG. 1106 shows the left ventricular outflow tract (shifted—3.9 mm from the four-chamber view), FIG. 1108 shows the right ventricular outflow tract (PA) (shifted—8.2 mm from the four-chamber view), and FIG. 1110 shows the three vessel view (shifted—10.9 mm from the four-chamber view). At step 5 in FIG. 2, images can also be automatically displayed in real time (or near real time), or displayed in a cineloop of a cardiac cycle with appropriate equipment.

In at least one embodiment of the invention, each of the standardized planes can be displayed automatically subsequent to acquisition of a reference plane within a volume. Once the mathematical and spatial relationships of the standardized volumes for a particular organ are established, then any standardized plane can serve as a reference plane (e.g., the aortic arch of the fetal heart). This is useful in obstetrical ultrasonography, given that the fetus may be in an orientation within the uterus allowing for only the aortic arch to be imaged on 2-D ultrasonography. One or more embodiments of the invention can then automatically display other standardized planes, such as the four-chamber view. Standardized planes can also be displayed for fetal organs other than the heart, as well as neonatal and adult organs.

In at least one embodiment of the invention, an image volume can be acquired with advanced transducers, and one or more planes of interest for a particular organ can be automatically displayed in real time upon acquisition. That is, the standard A, B and C planes do not need to be displayed prior to displaying one or more of the standard reference planes of interest. One or more reference planes of interest for a particular organ can thus be displayed directly from, and subsequent to, volume acquisition.

Due to the relatively small size of the fetus, 3-D and 4-D ultrasound obstetrical imaging allows for acquisition of multiple organs within a single 3-D volume. For example, a single 3-D volume of the fetal chest generally contains the heart, great vessels, venous connections to the heart and both lungs. At least one embodiment of the present invention therefore contemplates for a comprehensive, or substantially comprehensive, diagnosis or assessment of the fetal cardiovascular system from a single 3-D volume. When a volume that contains the entire fetus is acquired, the fetus can be re-oriented in a standardized, referenced position within the acquired volume. Then, any or all ultrasonographic standardized planes can be displayed, optionally automatically, to enable, for example, a physician to evaluate the fetal anatomy (e.g., head, chest, abdomen and/or extremities). Adult and neonatal organs can also be diagnosed in this manner.

At step 6, one or more embodiments of the present invention can utilize, for example, standard (e.g., off-the-shelf) image recognition software to assess the level of the standardized planes and diagnose, or facilitate diagnosis of, an imaged organ. For example, gray scale pattern recognition can be used to ensure proper orientation of automatically generated standardized planes and to compare a specific image (e.g., of and/or within the fetal heart) to one or more respective reference images. The gray scale pattern recognition comparison can be used to identify, for example, normal and abnormal anatomical structures and/or portions thereof. In the case of the fetal heart, the size of ventricles and/or outflow tracts can be compared with one or more corresponding reference images of ventricles and/or outflow tracts. A report can be generated that provides, for example, an indication of normal and abnormal relationships. One or more embodiments of the present invention can thus determine the location of fetal cardiac structures, such as the ventricles and/or the great vessels, and optionally provide data pertaining, for example, to the size and/or shape of structures and relative relationships. Adult and neonatal organs can also be diagnosed in this manner.

The many features and advantages of the invention are apparent from the detailed specification, and thus, it is intended to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention. While the foregoing invention has been described in detail by way of illustration and example of preferred embodiments, numerous modifications, substitutions, and alterations are possible.

1. A computer program product residing on a computer readable medium, for use in a medical-imaging environment, the computer program product comprising instructions for enabling a computer to:
acquire ultrasound image data for at least a portion of a body organ;
utilize data defining a reference plane for the body organ to define at least one other plane with respect to the reference plane; and
display automatically and substantially simultaneously at least two ultrasound images corresponding to at least one of the reference plane and the data defining the at least one other plane.

2. The computer program product according to claim 1, wherein the body organ is a fetal heart.

3. The computer program product according to claim 2, wherein the reference plane is a four-chamber view.

4. The computer program product according to claim 2, wherein the data defining the at least one other plane comprises data defining at least one of: a right ventricular outflow tract image, a left ventricular outflow tract image, a ductal arch image, an aortic arch image, a venous connections image, and a three vessel view image.

5. The computer program product according to claim 1, wherein the organ is a fetal head.

6. The computer program product according to claim 5, wherein the reference plane is a biparietal diameter of the fetal head.

7. The computer program product according to claim 1, wherein the processing by the computer is associated with sonography equipment.

8. The computer program product according to claim 1, wherein the instructions are executed by a general purpose computer.

9. The computer program product according to claim 1, further comprising instructions for causing the computer to provide a medical evaluation of the imaged organ.

10. The computer program product according to claim 9, wherein image recognition software is used to facilitate at least one of location of standardized planes and the medical evaluation.

11. The computer program product according to claim 9, wherein the medical evaluation comprises the steps of:
recognizing a specific structure within an image;
comparing the structure to a reference image; and
identifying at least one of normal and abnormal anatomical characteristics of the structure.

12. The computer program product according to claim 1, wherein the display of the at least two ultrasound images comprises for each image sagittal, transverse and coronal planes.

13. The computer program product according to claim 12, wherein the display is a real time display.

14. The computer program product according to claim 1, wherein the display of the at least two ultrasound images comprises a display of a single plane associated with each of the at least one other plane.

15. The computer program product according to claim 1, wherein the display of the at least two ultrasound images comprises a real time display, of one or more standardized planes, directly from a real time volume acquired at a reference level.

16. A method comprising:
acquiring ultrasound image data for at least a portion of a body organ;
utilizing data defining a reference plane for the body organ to define at least one other plane with respect to the reference plane; and
displaying automatically and substantially simultaneously at least two ultrasound images corresponding to at least one of the reference plane and the data defining the at least one other plane.

17. A system comprising:
a transducer for acquiring ultrasound image data for at least a portion of a body organ;
a processor for processing the ultrasound image data to define a reference plane for the body organ and define at least one other plane with respect to the reference plane; and
a display, wherein said processor facilitates displaying substantially simultaneously at least two ultrasound images corresponding to at least one of the reference plane and the data defining the at least one other plane.
